(19) United States
(12) Patent Application Publication
(10) Pub. No.: US 2024/0124557 A1
(43) Pub. Date: Apr. 18, 2024

(54) METHODS FOR DELIVERING ANTIBODIES TO THE FETAL CIRCULATION

(71) Applicant: The Children's Medical Center Corporation, Boston, MA (US)

(72) Inventor: Dario O. FAUZA, Boston, MA (US)

(73) Assignee: The Children's Medical Center Corporation, Boston, MA (US)

(21) Appl. No.: 18/392,902

(22) Filed: Dec. 21, 2023

Related U.S. Application Data
(63) Continuation of application No. PCT/US2022/034261, filed on Jun. 21, 2022.
(60) Provisional application No. 63/213,234, filed on Jun. 22, 2021.
(60) Provisional application No. 63/316,048, filed on Mar. 3, 2022.

(51) Int. Cl. C07K 16/06; A61P 37/04

(52) U.S. Cl. CPC C07K 16/06; A61P 37/04; A61K 2039/54

(57) ABSTRACT

The invention features compositions and methods for delivering antibodies to the fetal circulation to be used for preventing and/or treating a fetal alloimmune disorder and/or to treat and/or prevent perinatal infections and/or to prevent neonatal necrotizing enterocolitis.

### CROSS-REFERENCE TO RELATED APPLICATIONS

This application is a continuation under 35 U.S.C. § 111(a) of PCT International Patent Application No. PCT/US2022/034261, filed Jun. 21, 2022, designating the United States and published in English, which claims priority to and the benefit of U.S. Provisional Application No. 63/213,234, filed Jun. 22, 2021, and U.S. Provisional Application No. 63/316,048, filed Mar. 3, 2022, the entire contents of each of which are incorporated by reference herein.

### BACKGROUND OF THE INVENTION

Delivery of immunoglobulins directly to the fetus is invasive and morbid, while indirect delivery via the mother is limited by poor and selective placental transfer. Nevertheless, the US has over 4 million pregnancies per year and fetuses in need of treatment could benefit from improved methods for immunoglobulin delivery. Thus, there remains a need for improved, minimally invasive methods for delivery of immunoglobulins to the fetus.

### SUMMARY OF THE INVENTION

As described below, the invention features minimally invasive methods for delivering antibodies to the fetal circulation to be used for preventing and/or treating a fetal alloimmune disorder and/or to treat and/or prevent perinatal infections.

In one aspect, the invention features a method for reducing a perinatal infection in a fetus or neonate. The method involves administering to the amniotic fluid surrounding the fetus an antibody or antigen binding fragment thereof. The fetus or newborn has or has a propensity to develop a perinatal infection. The method reduces the infection.

In embodiments, the infection is a viral infection or a microbial infection. In embodiments, the infection is a viral infection. In embodiments, the infection a cytomegalovirus (CMV), erythrovirus B19, Enteroviruses, hepatitis B virus, toxoplasmosis, rubella, herpes virus, varicella zoster virus (VZV), and/or human immunodeficiency virus (HIV) infection. In embodiments, the infection is a Bacteroides spp., Borrelia spp., Campylobacter spp., Chlamydia spp., Candida spp., Coccidioides spp., Enterococcus spp., Escherichia coli, Haemophilus spp., Listeria spp., Mycobacterium spp., Neisseria spp., Proteus spp., Salmonella spp., Staphylococcus spp., Streptococcus spp., Toxoplasma spp., Treponema spp., and/or Veillonella spp. infection.

In embodiments, the administration is associated with a reduction in incidence of a surgical disease in the neonate. In embodiments, the surgical disease is necrotizing enterocolitis.

In embodiments, the administration is associated with a reduction in incidence of neonatal infection and/or severity of infection.

In one aspect, the invention features a method for treating an alloimmune disorder in a developing fetus. The method involves administering to the amniotic fluid surrounding a developing fetus an antibody or antigen binding fragment thereof, thereby treating the alloimmune disorder. In embodiments, the alloimmune disorder is a hemolytic disorder.

In one aspect, the invention features a kit suitable for use in carrying out the method of any of the above aspects. The kit contains the antibody of any of the above aspects.

In any of the above aspects, the antibody is administered by intra-amniotic injection. In any of the above aspects, the antibody is derived from an IgA, IgD, IgE, IgG, IgM, IgY and/or IgW antibody. In any of the above aspects, the antibody is derived from human IgG. In any of the above aspects, the antibody is derived from human IgA. In any of the above aspects, the antibody is derived from human IgM. In any of the above aspects, the antibody is derived from human IgD. In any of the above aspects, the antibody is derived from human IgE. In any of the above aspects, the antibody is derived from human IgY. In any of the above aspects, the antibody is derived from human IgW. In any of the above aspects, the antibody is an (ab′)₂, F(ab)₂, Fab′, Fab, scFv, or Fv. In any of the above aspects, the antibody is a nanobody, a diabody, a triabody, a tetrabody, a minibody, or a camelid single domain antibody. In any of the above aspects, the antibody is a monoclonal or polyclonal antibody. In any of the above aspects, the antibody is a human antibody.

In any of the above aspects, the fetus is a mammalian fetus. In any of the above aspects, the fetus is a human fetus.

In any of the above aspects, the antibody is administered prior to 10 weeks of pregnancy. In any of the above aspects, the antibody is administered prior to 5 weeks of pregnancy. In any of the above aspects, the antibody is administered after 10 weeks of pregnancy.

In any of the above aspects, from about 25 μg to about 500 mg of the antibody is administered In any of the above aspects, the antibody contains an Fc domain containing one or more mutations at one or more positions corresponding to Thr 250, Met 252, Ser 254, Thr 256, Thr 307, Glu 380, Met 428, His 433, and/or Asn 434 of human IgG1.

In any of the above aspects, the administration is associated with the presence of and/or an increase in levels of the antibody in the serum of the fetus. In any of the above aspects, the administration is associated with the presence of and/or an increase in levels of the antibody in a tissue of the fetus. In any of the above aspects, the tissue contains thymus, spleen, brain, and/or bone marrow tissue. In any of the above aspects, about or at least about 1 mg/kg fetal weight of the antibody is delivered to the fetus.

The invention provides methods for delivering antibodies to the fetal circulation to be used for preventing and/or treating a fetal alloimmune disorder and/or to treat and/or prevent perinatal infections. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small or large molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change in the expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” (Ab) refers to an immunoglobulin molecule, or antigen binding fragment thereof, that specifically binds to, or is immunologically reactive with, a particular antigen. Accordingly, antibodies described herein include polyclonal, monoclonal, pooled, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments, as well as engineered antibodies, which include CrossMabs (e.g., CrossMab^{Fabs}, CrossMab^{CH1-CL} and CrossMab^{VH-VL} formats). Moreover, unless otherwise indicated, the term “monoclonal antibody” (mAb) is meant to include both intact molecules, as well as, antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference). In one embodiment, an antibody is a biparatopic antibody.

By “antigen” is meant an agent to which an antibody specifically binds. Exemplary antigens include small molecules, carbohydrates, proteins, and polynucleotides.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “disease” is meant any condition or disorder that damages or interferes with the normal function of a cell, tissue, or organ. In embodiments the disease is associated with an infection, where the infection is optionally a viral infection or a microbial infection. Non-limiting examples of viral infections include those associated with cytomegalovirus (CMV), erythrovirus B19, Enteroviruses, hepatitis B virus, rubella, herpes virus, varicella zoster virus (VZV), and/or human immunodeficiency virus (HIV). Non-limiting examples of microbial infections include those associated with Bacteroides spp., Borrelia spp. (e.g., Borrelia burgdorferi), Campylobacter spp. (e.g., Campylobacter fetus), Chlamydia spp. (e.g., Chlamydia trachomatis), Candida spp. (e.g., Candida albicans), Coccidioides spp. (e.g., Coccidioides immitis), Enterococcus spp., Escherichia coli, Haemophilus spp., Listeria spp. (e.g., Listeria monocytogenes), Mycobacterium spp. (e.g., Mycobacterium tuberculosis), Neisseria spp. (e.g., Neisseria gonorrhoeae), Proteus spp., Salmonella spp. (e.g., Salmonella typhosa), Staphylococcus spp. (e.g., Staphylococcus aureus), Streptococcus spp. (e.g., Streptococcus pyogenes, Streptococcus agalactiae), Toxoplasma spp. (e.g., Toxoplasma gondii), Treponema spp. (e.g., Treponema pallidum), and/or Veillonella spp. In an embodiment, the disease is toxoplasmosis. In embodiments, the disease is an alloimmune disorder (e.g., an alloimmune hemolytic disorder). Further non-limiting examples of diseases are surgical diseases (e.g., necrotizing enterocolitis), where the surgical disease is optionally associated with an infection. In embodiments, the infections are perinatal infections.

By “effective amount” is meant the amount of an agent required to ameliorate the symptoms of a disease relative to an untreated patient. The effective amount of active compound(s) used to practice the invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fetus” is meant an unborn child.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, or more nucleotides or amino acids.

By “increase” is meant to alter positively by at least 1%. An increase may be by 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively by at least 1%. A reduction may be by 5%, 10%, 25%, 30%, 50%, 75%, or even by 100%.

By “reference” is meant a standard or control condition. In embodiments, the reference is a healthy subject.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, swine, leporine, or feline. In embodiments, the human is a fetus, neonate, a perinatal animal, or unborn infant.

By “surgical disease” is meant a disease that may be treated using surgery.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a box plot showing the median and interquartile range of IgG concentrations found in fetal serum from each intra-amniotic concentration group, as well in maternal serum from all three groups. Pairwise comparisons not statistically significant (p=0.064 to 0.121, Table 1).

FIG. 2 provides a box plot showing the median and interquartile range of IgG concentrations found in fetal bone marrow from each intra-amniotic concentration group. Refer to Table 1 for pairwise comparisons.

FIG. 3 provides a box plot showing the median and interquartile range of IgG concentrations found in fetal spleen from each intra-amniotic concentration group. Refer to Table 1 for pairwise comparisons.

FIG. 4 provides a box plot showing the Median and interquartile range of IgG concentrations found in fetal brain from each intra-amniotic concentration group. Refer to Table 1 for pairwise comparisons.

FIG. 5 provides a box plot showing the median and interquartile range of IgG concentrations found in fetal thymus from each intra-amniotic concentration group. All pairwise comparisons statistically significant (p=0.002 to p<001, Table 1).

FIG. 6 provides a collection of box plots showing human IgG levels (ng/mg) on gestational days 19, 20 and 21 after transamniotic injection of the IgG on day 18 (p=0.792 to <0.001 in different pairwise comparisons). All sample sites showed significantly lower IgG levels at E21 than E19 (p=0.010 to <0.001).

FIG. 7 provides a collection of box plots showing human IgG levels (ng/mg) in fetal serum, liver, stomach aspirate and amniotic fluid on gestational days 19, 20 and 21 after transamniotic injection on day 18 (p=0.505 to <0.002 in different pairwise comparisons).

FIG. 8 provides a collection of box plots showing human IgA levels at different fetal and gestational sites and time points after transamniotic administration.

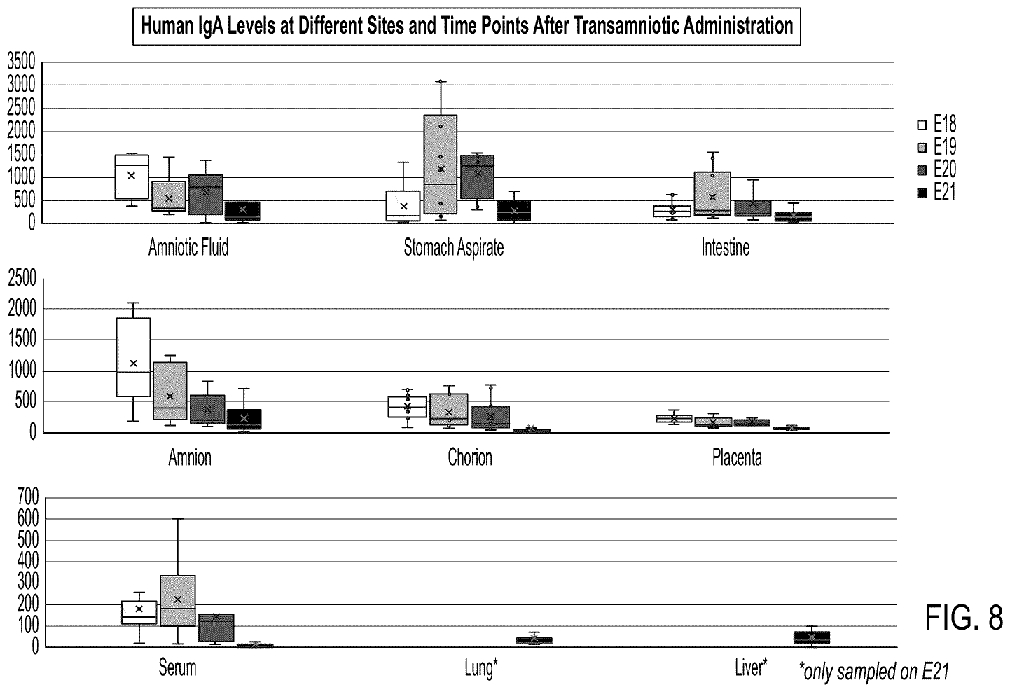

### DETAILED DESCRIPTION OF THE INVENTION

The invention features compositions and methods for delivering antibodies to the fetal circulation to be used for preventing and/or treating a fetal alloimmune disorder (e.g., an alloimmune hemolytic disorder) and/or to treat and/or prevent perinatal infections (e.g., a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection).

The invention is based, at least in part, upon the discovery that the amniotic fluid/cavity constitutes a practicable, minimally invasive access window to the fetal circulation. In the Examples provided herein, it is demonstrated that the amniotic cavity/fluid is a route for administration of therapeutic antibodies to the fetus and neonate. The Examples provided herein demonstrate that IgG antibodies can reach high levels in the fetal/neonatal circulation after simple intra-amniotic administration in a healthy rodent model. Delivery of antibodies to the fetus by intra-amniotic injection (termed Transamniotic Fetal Immunotherapy (TRAFIT)), therefore, is a viable strategy for the perinatal management of select diseases (e.g., alloimmune hemolytic disorder, necrotizing enterocolitis, cytomegalovirus (CMV), toxoplasmosis, rubella, herpes virus, and human immunodeficiency virus (HIV)).

As mentioned above, the invention is also based at least in part on the discovery that the transamniotic route was recently discovered as a minimally invasive means of fetal immunoglobulin administration, however by unclear mechanisms. As described further in the Examples provided below, experiments were undertaken to examine IgG routing after intra-amniotic delivery. Sprague-Dawley fetuses (n=78) received intra-amniotic injections of 15 mg/mL of human IgG on gestational-day 18 (E18; term=21-22 days). Amniotic fluid, amnion, chorion, placenta, fetal serum, liver, and stomach-aspirate samples were procured on E19, E20, and E21 for IgG quantification by ELISA. Statistical analysis was by median regression with Bonferroni-adjusted significance at p<0.017. Human IgG was detected at all sampled sites across all time points, though at significantly higher levels in the gestational membranes and fetal serum than in the stomach aspirate and liver (p<0.001 for both). Gestational membranes showed a daily decrease after injection, stabilizing by E20 and E21 (p=0.792 to <0.001). Placental levels were significantly lower at E21 than E19 (p=0.010). Fetal serum showed the highest human IgG levels at term. The chronology of exogenous IgG kinetics after intra-amniotic injection was suggestive of direct placental transport leading to consistently high fetal serum levels, possibly combined with some fetal ingestion. Transamniotic fetal immunotherapy (TRAFIT) may become a practicable strategy for the prenatal treatment of select alloimmune disorders and infections.

### Intra-Amniotic Delivery

The invention provides trans-amniotic fetal immunotherapy (TRAFIT) methods for treating a fetal alloimmune disorder (e.g., an alloimmune hemolytic disorder) and/or treating and/or preventing perinatal infections (e.g., a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection) and/or preventing necrotizing enterocolitis or symptoms thereof. Thus, one embodiment is a method of treating a fetus suffering from or susceptible to a disease (e.g., necrotizing enterocolitis), infection, and/or disorder or symptom thereof. The methods involve administering to the amniotic fluid a therapeutically effective amount of an antibody. The antibody is administered by intra-amniotic injection. The method involves the step of administering to the fetus a therapeutic amount of an amount of an antibody sufficient to treat the disease or disorder or symptom thereof, under conditions such that the disease, infection, and/or disorder is treated and/or prevented.

In embodiments, the mother within which the fetus is developing has the infection. In embodiments, the mother has a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection. In a particular embodiment, administration of the antibody to the amniotic fluid prevents or reduces transfer of the infection from the mother to the fetus.

The methods herein include administering to the amniotic fluid surrounding a fetus an effective amount of an antibody, or a composition described herein to produce such effect. Identifying a subject in need of such treatment can be in the judgment of a subject or a health care professional and can be subjective (e.g., opinion) or objective (e.g., measurable by a test or diagnostic method).

The therapeutic methods of the invention (which include prophylactic treatment) in general comprise administration of a therapeutically effective amount of an antibody to the amniotic fluid surrounding a fetus. In embodiments, the fetus is a human fetus. Such treatment will be suitably administered to fetuses, suffering from, having, susceptible to, or at risk for a disease, infection, and/or disorder, or symptom thereof, before and/or after birth. Determination of those subjects “at risk” can be made by any objective or subjective determination by a diagnostic test or opinion of a subject or health care provider (e.g., genetic test, enzyme or protein marker, Marker (as defined herein), family history, and the like).

In some embodiments, an antibody is administered to the amniotic fluid in an amount of about or at least about 1 μg, 5 μg, 10 μg, 15 μg, 20 μg, 25 μg, 100 μg, 250 μg, 500 μg, 750 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 100 mg, 200 mg, 300 mg, 400 mg, 500 mg, 600 mg, 700 mg, 800 mg, 900 mg, or 1,000 mg. In some embodiments, an antibody is administered to the amniotic fluid in an amount of no more than about 1 μg, 5 μg, 10 μg, 15 μg, 20 μg, 25 μg, 100 μg, 250 μg, 500 μg, 750 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 200 mg, 300 mg, 400 mg, 500 mg, 600 mg, 700 mg, 800 mg, 900 mg, or 1,000 mg.

In some embodiments, administration of an antibody of the invention to the amniotic fluid is associated with a reduction in the intensity, severity, or frequency, or delays the onset of an alloimmune disorder (e.g., an alloimmune hemolytic disorder), disease, and/or infection (e.g., a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection) in the fetus before and/or after birth (i.e., prenatal or perinatal).

It can be advantageous to administer the antibody at a particular time during the development of the fetus. In embodiments, the antibody is administered prior to, at, after, and/or until about 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 25 weeks, 30 weeks, 35 weeks, or 40 weeks of pregnancy. In embodiments, it is advantageous to administer the antibody prior to 20 weeks of pregnancy.

In embodiments, the trans-amniotic delivery of the antibody to the amniotic fluid results in the presence of and/or an increase in levels of the antibody in the fetal blood plasma. In embodiments, the trans-amniotic delivery of the antibody to the amniotic fluid results in an increase in levels of the antibody in fetal tissues (e.g., thymus, spleen, brain, and/or bone marrow, and/or others, given the presence of the antibody in the fetal blood plasma).

A skilled practitioner will readily be able to identify suitable methods for detecting and measuring levels of an antibody in the amniotic fluid or in any samples taken from a fetus or from a neonate that received such treatment before birth. For example, ELISA is one example of many techniques available for measuring antibody levels in biological samples such as, to provide non-limiting examples, serum, bone marrow, spleen tissue, thymus tissue, and brain tissue. The biological samples can be collected from the fetus and/or the mother.

### Antibodies

The methods of the invention involve administering an antibody to the amniotic fluid surrounding a fetus.

Non-limiting examples of antibody compositions suitable for use in the methods and compositions of the invention include those comprising CMV-targeted IgG's (e.g., Cytogam and Cytotect CP), hepatitis B-targeted IgG's (e.g., Hepatect and Zutectra CP), Varicella zoster IgG's (e.g., Veritect CP), and anti-HIV IgG's (e.g., HIVIG, which is disclosed in Stiehm, et al., for the Pediatric AIDS Trials Group Protocol 273 Study Group, Use of Human Immunodeficiency Virus (HIV) Human Hyperimmune Immunoglobulin in HIV Type 1-Infected Children (Pediatric AIDS Clinical Trials Group Protocol 273), The Journal of Infectious Diseases, Volume 181, Issue 2, February 2000, Pages 548-554). Cytogam (concentration of 50±50 mg immunoglobulin/ml) is produced by CSL Berhling, Bern Switzerland and the globulin is stabilized with 5% sucrose and 1% human albumin (see Snydman D R, McIver J, Leszczynski J, et al. A pilot trial of a novel cytomegalovirus immune globulin in renal transplant recipients. Transplantation 1984; 38:553-557). Cytoprogect CP is produced by Biotest, Dreieich Germany and contains about 500 mg human plasma protein (of which about 96% is immunoglobulin G), with a content of antibodies against CMV of about 1,000 U. Hepatect CP is also produced by Biotest and contains about 50 g/l of 96% IgG with a content of antibodies to Hepatitis B virus surface antigen (HBs) of about 50 IU/ml. Zutectra CP is also produced by Biotest and is similar to Hepatect CP. Varitect CP, which is also produced by Biotest, comprises about 25 IU/ml of anti-Varicella zoster human immunoglobulin. HIVIG is produced by North American Biologicals, Boca Raton, FL and contains a 5% solution (50 mg/mL) containing >98% plus dimeric IgG and an anti-p24 titer of 1:160,000.

Further non-limiting examples of antibody compositions suitable for use in the methods of the invention include those containing antibodies suitable for use in intravenous immunoglobulin therapy (IVIg), such as Bivigam (ADMA Biologics Ramsey, NJ USA), Carimune (CSL Gerhling, Bern Switzerland; available as a powder), Flebogamma DIF (Grifols, Barcelona Spain), Gammagard S/D (Takeda Pharmaceuticals, Deerfield, IL USA; containing less than lmcg/ml IgA in 5% solution, containing glucose in solution, and available as a powder), Gammagard Liquid (Takeda Pharmaceuticals, Deerfield, IL), Gammaked (Kedrion Biopharma, Fort Lee, NJ USA), Gammaplex (Bioproducts limited, Hertfordshire UK; having a low IgA content), Gamunex (Grifols, Barcelona Spain), Octagam (Octapharma, Paramus, NJ), and Privigen (CSL Berhling, Bern Switzerland).

Additional non-limiting examples of antibody compositions suitable for use in the methods of the invention include immunoglobulin mixes with IgG, such as Pentaglobin (Biotest, Dreieich Germany) and Trimodulin (Biotest, Dreieich Germany). Pentaglobin contains IgA and IgM and has been used in sepsis, pneumonias, and to treat gastrointestinal infections (see, Borleffs J C, Schellekens J F, Brouwer E, Rozenberg-Arska M. Use of an immunoglobulin M containing preparation for treatment of two hypogammaglobulinemic patients with persistent Campylobacter jejuni infection. Eur J Clin Microbiol Infect Dis. 1993 October; 12(10):772-5). Trimodulin contains about 23% IgM, about 21% IgA, and about 56% IgG (see, Welte T, Dellinger R P, Ebelt H, Ferrer M, Opal S M, Singer M, et al. Efficacy and safety of trimodulin, a novel polyclonal antibody preparation, in patients with severe community-acquired pneumonia: a randomized, placebo-controlled, double-blind, multicenter, phase II trial (CIGMA study). Intensive Care Med. 2018; 44(4):438-48).

In embodiments, the antibody compositions of the invention contain 1%, 2%, 3%, 4%, 5%, 6%, 7%, 8%, 9%, 10%, 11%, 12%, 13%, 14%, 15%, or 20% (wt/wt) of the antibody.

Antibodies of all classes can be made by any of the methods known in the art. One method of obtaining antibodies is to immunize suitable host animals with an immunogen and to follow standard procedures for polyclonal or monoclonal antibody production. The immunogen will facilitate presentation of the immunogen on the cell surface. In embodiments, the immunogen is associated with an infectious agent associated with an infection (e.g., a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection). Immunization of a suitable host can be carried out in a number of ways. Nucleic acid sequences encoding a polypeptide of interest, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised. In some embodiments the antibody is IgG.

In embodiments, the antibody contains an Fc domain or a portion thereof that binds to the FcRn receptor. As a non-limiting example, a suitable Fc domain may be derived from an immunoglobulin subclass such as IgG. In some embodiments, a suitable Fc domain is derived from IgG1, IgG2, IgG3, or IgG4. Suitable Fc domains include those derived from human or humanized antibodies.

Not being bound by theory, improved binding between Fc domain and the FcRn receptor results in prolonged serum half-life. Thus, in some embodiments, a suitable Fc domain comprises one or more amino acid mutations that lead to improved binding to FcRn. Various mutations within the Fc domain that effect improved binding to FcRn are known in the art and can be adapted to practice the invention. In some embodiments, a suitable Fc domain comprises one or more mutations at one or more positions corresponding to Thr 250, Met 252, Ser 254, Thr 256, Thr 307, Glu 380, Met 428, His 433, and/or Asn 434 of human IgG1. Non-limiting examples of methods for increasing the serum half-life of an immunoglobulin are provided in Longer serum halflife: Hinton P R, Xiong J M, Johlfs M G, et al. An engineered human IgG1 antibody with longer serum half-life. J Immunol 2006; 176 (1):346-56.

Antibodies against a polypeptide of interest may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ antibody proteins on its surface. Genes of antibody gene libraries are inserted into a population of phage. Each phage carries the genes for a different antibody and thus displays a different antibody on its surface.

Antibodies made by any method known in the art can then be purified from a host cell. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

### Pharmaceutical Compositions

The invention provides compositions comprising an antibody of any class and type for use in methods for preventing and/or treating a fetal alloimmune disorder (e.g., an alloimmune hemolytic disorder) and/or to treat and/or prevent perinatal infections (e.g., a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection) and/or prevent necrotizing enterocolitis. The compositions should be sterile and contain a therapeutically effective amount of the antibody in a unit of weight or volume suitable for administration to a subject.

Agents (e.g., antibodies) of the invention may be administered within a pharmaceutically-acceptable diluent, carrier, or excipient, in unit dosage form. Conventional pharmaceutical practice may be employed to provide suitable formulations or compositions to administer an antibody to amniotic fluid. Therapeutic formulations may be in a liquid form (e.g., liquid solutions or suspensions) or in a solid form (e.g., a lyophilized composition or powder).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interfere with their activity. In an embodiment, a water-soluble carrier suitable for intravenous administration is used.

A suitable pharmaceutical composition, if desired, can also contain minor amounts of wetting or emulsifying agents, or pH buffering agents. A composition can be a liquid solution, suspension, emulsion, tablet, pill, capsule, sustained release formulation, or powder.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful delivery systems for agents of the invention include ethylene-vinyl acetate copolymer particles, osmotic pumps, implantable infusion systems, and liposomes.

The formulations can be administered to human patients in therapeutically effective amounts. The preferred dosage of an antibody the invention is likely to depend on such variables as the infection being treated, volume of the amniotic cavity, or the nature of a disorder being treated. In an embodiment, the antibody is administered more than once in a given pregnancy.

A pharmaceutical composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline, or dextrose/water. Where the composition is administered by injection, an ampule of sterile water for injection or saline can be provided so that the ingredients may be mixed prior to administration.

Generally, doses of antibodies of the invention will be from about 0.01 mg/kg fetal weight per day to about 1000 mg/kg fetal weight per day and/or per administration. It is expected that doses ranging from about 1 to about 50 mg/kg will be suitable. In an embodiment, an antibody is administered to the amniotic fluid in amounts sufficient to deliver about or at least about 0.1 mg/kg fetal weight, 0.5 mg/kg fetal weight, 1 mg/kg fetal weight, 2 mg/kg feta weight, 3 mg/kg feta weight, 4 mg/kg feta weight, 5 mg/kg feta weight, 6 mg/kg feta weight 7 mg/kg feta weight, 8 mg/kg feta weight, 9 mg/kg feta weight, or 10 mg/kg fetal weight of the antibody to the fetus. In an embodiment, an antibody is administered to the amniotic fluid in amounts sufficient to deliver no more than about 1 mg/kg fetal weight, 2 mg/kg feta weight, 3 mg/kg feta weight, 4 mg/kg feta weight, 5 mg/kg feta weight, 6 mg/kg feta weight 7 mg/kg feta weight, 8 mg/kg feta weight, 9 mg/kg feta weight, or 10 mg/kg fetal weight of the antibody to the fetus. In the event that a response in a subject is insufficient at the initial doses applied, higher doses (or effectively higher doses) may be employed to the extent that subject tolerance permits. Multiple doses per day are contemplated to achieve appropriate systemic levels of an agent and/or compositions of the invention.

### Kits

The invention provides kits for use in preventing and/or treating a fetal alloimmune disorder (e.g., an alloimmune hemolytic disorder) and/or to treat and/or prevent perinatal infections (e.g., a cytomegalovirus (CMV), erythrovirus B19, Enteroviruses, hepatitis B virus, rubella, herpes virus, varicella zoster virus (VZV), toxoplasmosis, and/or human immunodeficiency virus (HIV) infection) and/or prevent necrotizing enterocolitis. In one embodiment, the kit contains a pharmaceutical composition containing an antibody suitable for preventing and/or treating the fetal alloimmune disorder and/or perinatal infection and/or necrotizing enterocolitis. In an embodiment, the kit contains equipment (e.g., hypodermic needles and syringes) to aid in administration of compositions of the invention to amniotic fluid surrounding a fetus.

Optionally, the kit includes directions for administering the pharmaceutical composition to amniotic fluid. In other embodiments, the kit comprises a sterile container which contains the pharmaceutical composition. Such containers can be boxes, ampules, bottles, vials, tubes, bags, pouches, blister-packs, or other suitable container form known in the art. Such containers can be made of plastic, glass, laminated paper, metal foil, or other materials suitable for holding compositions containing an antibody. The instructions will generally include information about the pharmaceutical composition (e.g., safety information, recommended doses, and the like) and how to administer the composition to the amniotic fluid surrounding a fetus. In other embodiments, the instructions include at least one of the following: description of the antibody; methods for using the enclosed materials; precautions; warnings; indications; clinical or research studies; and/or references. The instructions may be printed directly on the container (when present), or as a label applied to the container, or as a separate sheet, pamphlet, card, or folder supplied in or with the container.

The practice of the invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Survival

As a first step in the evaluation of the feasibility of delivering antibodies to the fetus by intra-amniotic injection, the impact of intra-amniotic injections of antibodies on survival was evaluated. Overall fetal survival to term was 83% (90/109), with no significant difference between groups, which were distributed as follows: 18 fetuses in the 5 mg/mL group, 24 in the 10 mg/mL group, 23 in the 15 mg/mL group, and 25 in the saline group. Of the 25 survivors in the saline group, 14 were randomly selected for IgG detection as controls for lack of human-rodent homology. Maternal survival was 100%.

### Example 2: IgG Presence in Fetal Serum

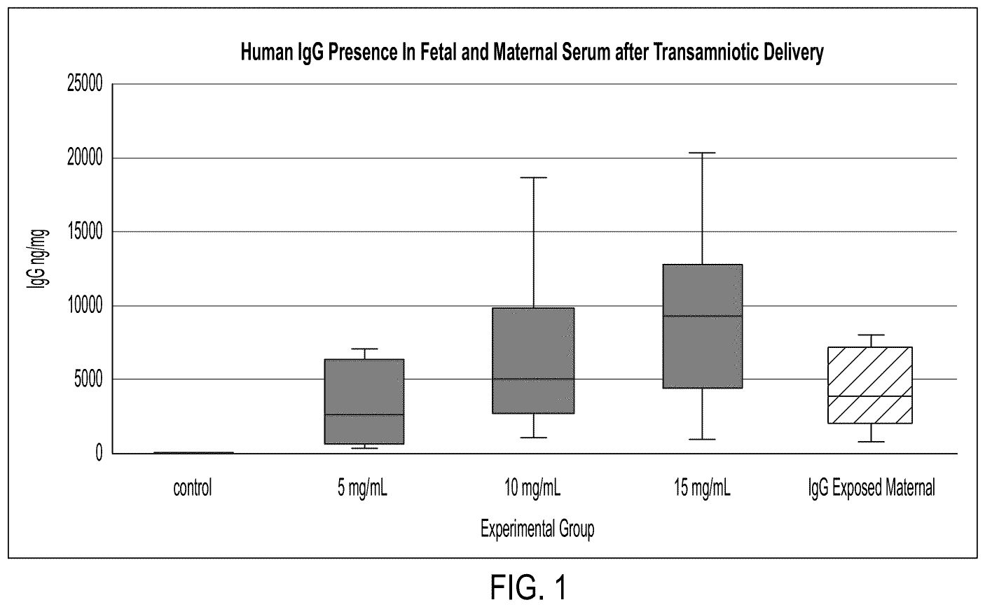

Next, it was determined whether or not an antibody delivered to the amniotic fluid was taken up by the fetus and present in the serum of the fetus. Human IgG was detected in the serum of fetuses from all three IgG injection concentration groups, but in none from the saline injection group (p<0.001, FIG. 1). A trend towards a direct correlation between the levels of human IgG in fetal serum and the concentration of intra-amniotic IgG injection was detected (Table 1). Human IgG was also detected in the serum of all 6 dams that underwent antibody injections (FIG. 1).

TABLE 1
Human IgG concentrations in each fetal sample stratified
and compared by intra-amniotic injection concentration.

| | IgG | IgG | IgG | |
|---|---|---|---|---|
| Sample | Injection | Presence (ng/mL) - | Injection | |
| Type | Concentration | Median (IQR) | Comparisons | p Value |
| Serum | 5 mg/mL | 2620 (598, | 5 vs 10 | 0.121 |
| | (n = 18) | 6333) | mg/mL | |
| | 10 mg/mL | 5049 (2765, | 10 vs 15 | 0.194 |
| | (n = 24) | 9708) | mg/mL | |
| | 15 mg/mL | 9241 (4391, | 5 vs 15 | 0.064 |
| | (n = 23) | 12684) | mg/mL | |
| Thymus | 5 mg/mL | 1.4 (1.27, | 5 vs 10 | 0.002* |
| | (n = 18) | 1.72) | mg/mL | |
| | 10 mg/mL | 2.08 (1.45, | 10 vs 15 | <0.001* |
| | (n = 24) | 3.35) | mg/mL | |
| | 15 mg/mL | 4.52 (3.04, | 5 vs 15 | <0.001* |
| | (n = 23) | 6.66) | mg/mL | |
| Spleen | 5 mg/mL | 1.01 (0.71, | 5 vs 10 | 0.113 |
| | (n = 18) | 1.55) | mg/mL | |
| | 10 mg/mL | 1.43 (0.79, | 10 vs 15 | 0.017 |
| | (n = 24) | 4.29) | mg/mL | |
| | 15 mg/mL | 2.52 (1.73, | 5 vs 15 | <0.001* |
| | (n = 23) | 4.2) | mg/mL | |
| Bone | 5 mg/mL | 4.34 (1.93, | 5 vs 10 | <0.001* |
| Marrow | (n = 18) | 6.27) | mg/mL | |
| | 10 mg/mL | 3.23 (1.91, | 10 vs 15 | 0.004* |
| | (n = 24) | 5.32) | mg/mL | |
| | 15 mg/mL | 7.27 (5.23, | 5 vs 15 | 0.021 |
| | (n = 23) | 8.87) | mg/mL | |
| Brain | 5 mg/mL | 0.91 (0.59, | 5 vs 10 | 0.063 |
| | (n = 18) | 1.61) | mg/mL | |
| | 10 mg/mL | 1.45 (0.95, | 10 vs 15 | 0.706 |
| | (n = 24) | 2.29) | mg/mL | |
| | 15 mg/mL | 1.54 (0.92, | 5 vs 15 | 0.118 |
| | (n = 23) | 1.76) | mg/mL | |

*Statistically significant, Bonferroni-adjusted at p < 0.008.

### Example 3: IgG Presence in Fetal Tissue

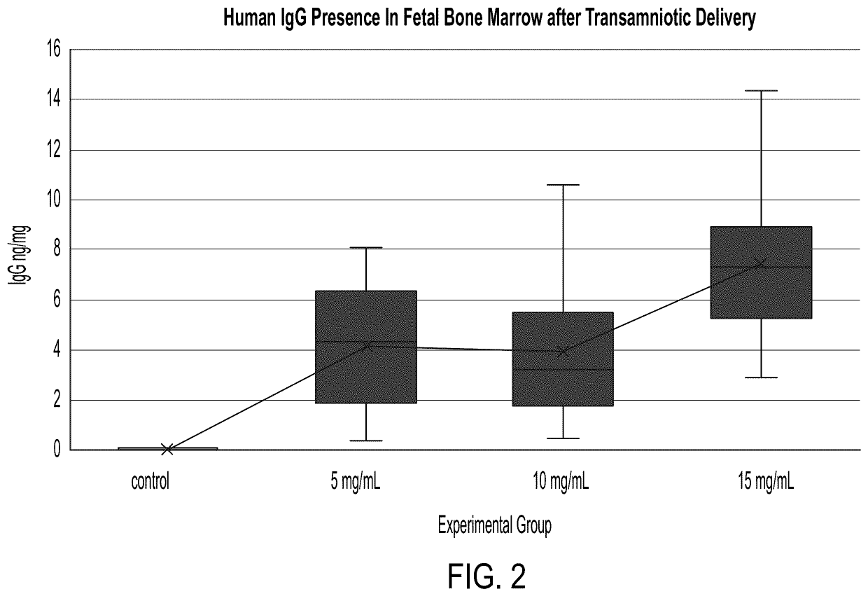

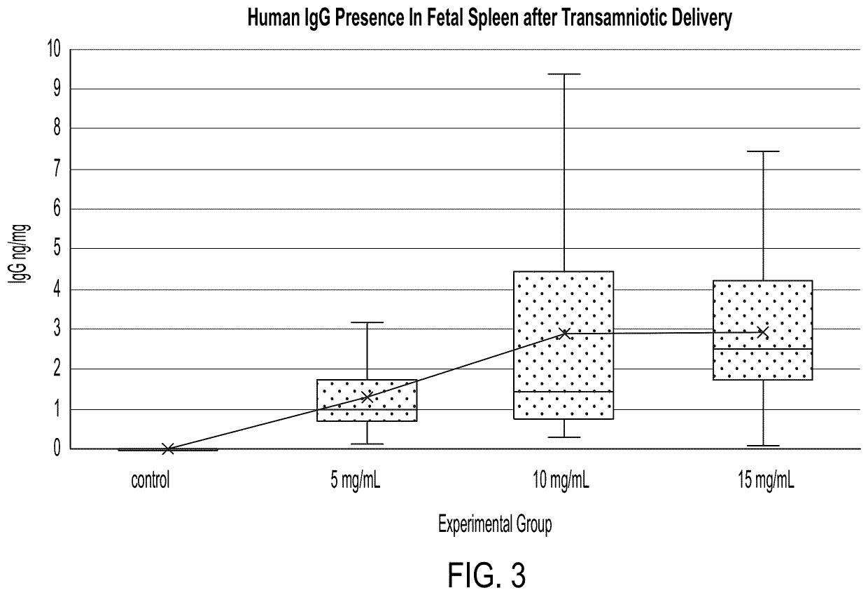

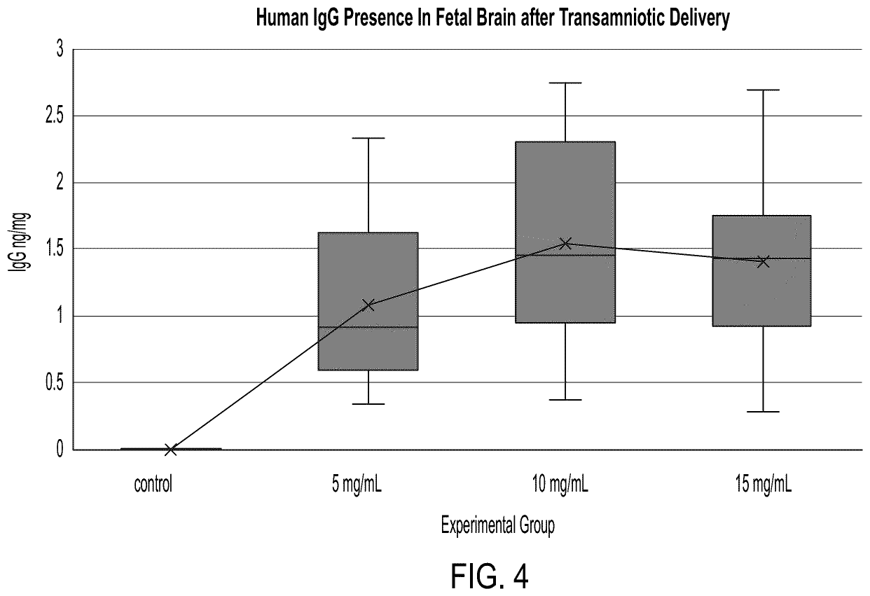

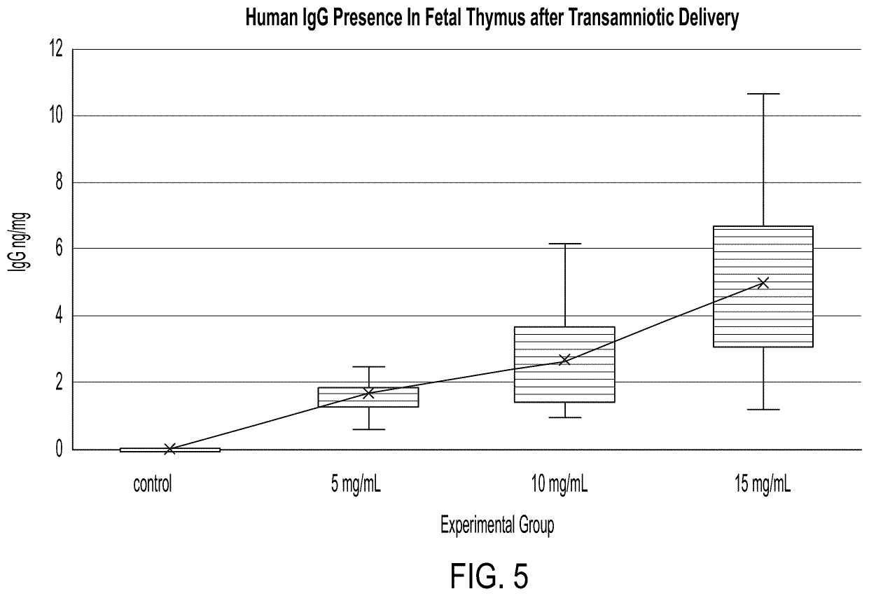

Finally, it was determined whether an antibody delivered to the amniotic fluid was taken up by the fetus and delivered to fetal tissues. Human IgG was detected in all fetal tissue specimens analyzed (thymus, spleen, brain, and bone marrow), albeit at lower levels than those detected in the fetal serum (Table 1, FIGS. 2-5). Also here, none of the tissue samples from the saline injection group had IgG present (p<0.001). A statistically significant dose dependent relationship between local IgG levels and IgG injection concentration at all three injection concentration groups (Table 1, FIG. 3) was observed in the thymus. In the spleen and bone marrow, such a correlation was observed at some injection concentrations (Table 1).

### DISCUSSION

The above-provided examples demonstrated that amniotic fluid is a reliable route of administration of antibodies to the fetus (and mother). Not wishing to be bound by theory, IgG is the predominant immunoglobulin in fetal life. Fetal immunity is heavily reliant on maternal transfer of immunoglobulin to fetal circulation via the placenta, as the fetus produces very low amounts of IgM and IgG throughout gestation. In humans, maternal transfer begins around 20 weeks of pregnancy, primarily with IgG, and sharply increases as gestation continues until reaching levels even higher than those seen in maternal serum. Some IgM also eventually passes to the fetus, while IgA, IgD, and IgE all do not cross the placenta.

Such limitations in antibody placental transfer contribute to a virtual absence of innate fetal immunity, rendering preterm infants especially vulnerable to infection as they have not received the full extent of passive immunization from the mother. Term infants are also at risk, especially those born to mothers with certain infections such as cytomegalovirus (CMV), for example, as they have already been exposed to the detrimental effects of the virus by the time the placental transfer of immunoglobulin starts to occur.

The present study demonstrates that transamniotic antibody delivery is an attainable strategy to enhance fetal and neonatal immunity. The timing and environment in which this novel strategy operates will likely transform the impact of passive fetal immunotherapy. For example, it will likely have an effect on in utero infections currently still associated with devastating consequences, such as CMV, toxoplasmosis, rubella, herpes virus, and even HIV. Notably, all of these in utero infections are known for their severe neurologic complications. Auspiciously, IgG was found in the brains of all fetuses in the above-presented examples. This strategy likely have clinical relevance to alloimmune disorders of pregnancy, such as alloimmune hemolytic disorders of the fetus.

The effects of transamniotic antibody delivery will likely extend well into the early neonatal period. Human IgG has a long half-life, of up to one month, which can be extended even further by modifications of its interaction with the FcRn receptor, found in the neonatal endothelium and intestinal lumen, which mediates the uptake and recycling of IgG and prevents lysosomal degradation (Hinton P R, Xiong J M, Johlfs M G, et al. An engineered human IgG1 antibody with longer serum half-life. J Immunol 2006; 176 (1):346-56). The methods provided herein likely lessen the burden of surgical disease (e.g., necrotizing enterocolitis).

The data presented in the Examples provided above supports the notion that transamniotic fetal immunotherapy (TRAFIT) is a practicable strategy for the perinatal management of select diseases. The choice for anatomical sites procured for analysis in this study derived from their relevance to the immune response and/or to their participation in the pathophysiology of disease process potentially amenable to TRAFIT. Not wishing to be bound by theory, a variety of paths may be at play in the uptake of the antibodies from the amniotic fluid by the fetus, including, as non-limiting examples, fetal aspiration and ingestion of amniotic fluid, direct placental uptake, and the immature fetal dermis.

### Example 4: Routing Kinetics of Human Immunoglobulin G after Transamniotic Fetal Immunotherapy (TRAFIT) in a Rodent Model

Fetal humoral immunity is constitutively depressed when compared with that of postnatal life. Prenatal administration of immunoglobulins by conventional means such as indirectly via the mother or direct fetal injections has proven ineffective and/or morbid. The transamniotic route has been recently shown experimentally to be a minimally invasive alternative for prenatal immunoglobulin administration, with at least immunoglobulins of the G class (IgG) reaching the fetal circulation and multiple anatomic sites after simple intra-amniotic injection (other classes are currently under investigation). This novel strategy, termed transamniotic fetal immunotherapy (TRAFIT), carries translational implications to a number of diseases, including possibly some in the pediatric surgery realm, such as necrotizing enterocolitis (for example, should IgGA also be able to be delivered to the fetal bowel via TRAFIT). Therefore, experiments were undertaken to determine the route through which exogenous IgG reaches the fetus after delivery into the amniotic fluid in a healthy rodent model.

Overall fetal survival to term was 86% (67/78), with no significant difference between the time-point groups. There were 20 surviving fetuses in the E19 group, 22 in the E20 group, and 25 in the E21 group. Maternal survival was 100%.

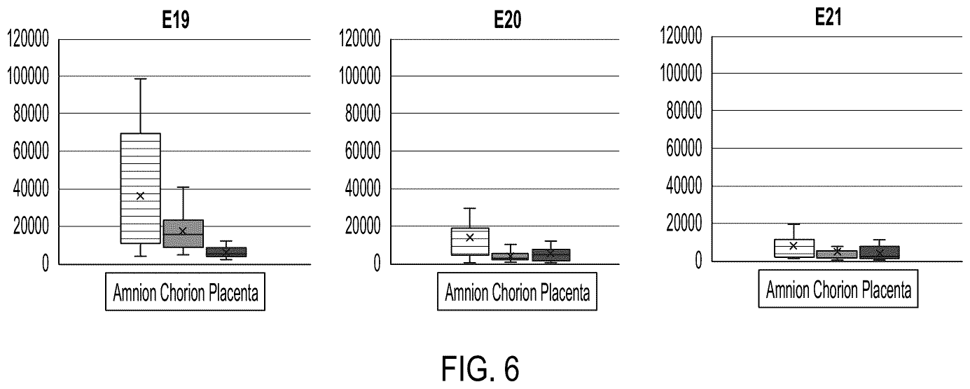

Human IgG was detected in the amniotic fluid, gestational membranes (amnion and chorion), and placenta at all time-points, with levels being significantly higher in the gestational membranes and placenta than in the amniotic fluid (p<0.001), in the fetal stomach aspirate (p<0.001), and in the fetal liver (p<0.001). Amniotic fluid IgG levels were statistically similar throughout all three time-points (p=0.028 to 0.151). Combined, the gestational membranes showed a daily decrease after injection, stabilizing by E20 and E21 (FIG. 6; p=0.792 to <0.001). Placental IgG levels were significantly lower at E21 than E19 (p=0.010).

Within the gestational membranes the amnion had the highest levels of human IgG at all time-points (p<0.001 for all comparisons). On E19, the chorion had higher levels than the placenta (p<0.001), but that was inverted on E20, when placental levels were significantly higher than chorionic levels (p<0.001). On E21 the amnion, chorion and placental all reached IgG levels between 3,101 ng/ml and 3,527 ng/ml.

As in the fetal annexes, human IgG was detected in the fetal serum, liver, and stomach aspirate across all time-points. The serum had the highest fetal levels, which peaked on E19 and stabilized on E20 to E21 (p=0.002 to 0.536). Stomach aspirate and amniotic fluid levels trended in parallel to each other, although, unlike the amniotic fluid, at E21 stomach aspirate levels were significantly lower than at both prior time-points in that site (p=0.009 and p=0.002, respectively;

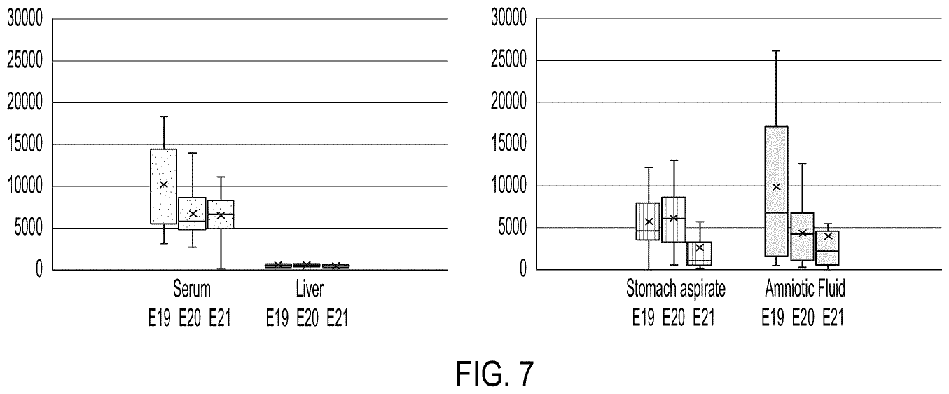

FIG. 7). Human IgG levels remained low in the fetal liver at all three time-points, with no appreciable differences between them (p=0.047 to 0.505). At term, the fetal serum showed the highest human IgG levels (6,762±5,024-7,699 ng/mg) of all fetal sites and was also notably higher than all gestational membranes, placenta, and amniotic fluid at this time-point (p<0.001 for all; Table 2; FIG. 7).

TABLE 2
Human IgG levels (ng/mg) at each sample site on E19, E20 and E21
after injection on E18. Data presented as median (IQR) values.

| | | | | Non-Nested: | | | Nested by Dam: Median | | |
|---|---|---|---|---|---|---|---|---|---|
| Comparison of Human IgG Present | | | | Mann-Whitney U test | | | Regression | | |
| on E19, E20, and E21 | | | | E19 vs | E19 vs | E20 vs | E19 vs | E19 vs | E20 vs |
| Organ | E19 | E20 | E21 | E20 | E21 | E21 | E20 | E21 | E21 |
| Amniotic | 6824 | 4255 | 2029 | 0.151 | 0.028* | 0.321 | 0.285 | 0.109 | 0.224 |
| Fluid | (1648, | (1759, | (586, | | | | | | |
| | 16980) | 4849) | 3813) | | | | | | |

Gestational Membranes

| | | | | | | | | | |
|---|---|---|---|---|---|---|---|---|---|
| Amnion | 22138 | 7943 | 3527 | 0.009** | <0.001** | 0.030* | 0.049* | 0.008** | <0.001** |
| | (12745, | (5062, | (2159, | | | | | | |
| | 62959) | 16554) | 7955) | | | | | | |
| Chorion | 16415 | 2942 | 3333 | <0.001** | <0.001** | 0.792 | <0.001** | <0.001** | 0.665 |
| | (9216, | (2289, | (1867, | | | | | | |
| | 22051) | 5502) | 4457) | | | | | | |
| Placenta | 5948 | 5109 | 3101 | 0.199 | 0.010** | 0.348 | 0.874 | <0.001** | 0.467 |
| | (4409, | (1895, | (1457, | | | | | | |
| | 8214) | 7084) | 7025) | | | | | | |

Fetal Samples

| | | | | | | | | | |
|---|---|---|---|---|---|---|---|---|---|
| Stomach | 4612 | 6065 | 1033 | 0.693 | 0.009** | 0.002** | 0.136 | 0.011** | <0.001** |
| Aspirate | (3621, | (3520, | (531, | | | | | | |
| | 7895) | 8478) | 3251) | | | | | | |
| Serum | 10555 | 5762 | 6762 | 0.008** | 0.002** | 0.536 | 0.106 | 0.202 | 0.004** |
| | (6715, | (4702, | (5024, | | | | | | |
| | 14585) | 7574) | 7699) | | | | | | |
| Liver | 743 | 730 | 651 | 0.505 | 0.047* | 0.088 | 0.103 | 0.007** | 0.027* |
| | (523, 789) | (551, 782) | (498, 704) | | | | | | |

*p < 0.05;
**Bonferroni-adjusted p < 0.017 (shown in bold).

Median regression controlling for dam of origin had no to minimal impact to these analyses, with only the fetal serum levels showing a significant increase from E20 to E21 (p=0.004) and the fetal liver levels showing a significant decrease from E19 to E21 (p=0.007) (Table 2).

The notion of TRAFIT as a therapeutic strategy for exogenous antibody delivery to the fetal circulation is arguably surprising, and perhaps because of that, a very recent development. One would predict that antibodies delivered to the amniotic fluid could, for example, easily reach the fetal lung via plain aspiration from fetal breathing movements, as it has been shown in so many other instances of a variety of substances or agents delivered to the amniotic fluid. Yet, the idea that there is a pathway from the fluid to the fetal blood is far from intuitive. This study starts to elucidate such a pathway.

After intra-amniotic injection of the human IgG, high levels were immediately detected in the amnion, chorion, and placenta, in parallel to high levels in the fetal serum. These levels then dropped off daily in a fairly synchronous fashion, supporting the presence of IgG absorption, and/or active transport via the gestational membranes and placenta before eventually being transferred to fetal serum. Free floating IgG has a half-life of approximately 21 days; therefore, proteolysis should not be the explanation for this drop, given the time frame of this rodent model. At the same time, IgG was also found in the stomach aspirate, following a pattern comparable to what was measured in the amniotic fluid. These findings are compatible with fetal swallowing possibly being an additional route of fetal IgG uptake, albeit at lower magnitude than what was observed through direct gestational membrane/placental transfer. Not intending to be bound by theory, one would postulate similar respiratory tract findings to those seen in the stomach aspirate.

Of note, these two apparent mechanisms of IgG routing—transplacental absorption and, possibly, fetal ingestion—have something in common: both the placenta and the fetal intestinal lumen harbor so-called neonatal fragment crystallizable (FcRN) receptors. Also commonly known as the neonatal IgG receptor despite its documented fetal presence and persistence throughout postnatal life, the FcRN receptor is the major driver of IgG uptake and recycling in normal physiology. It's location on the placental endothelium allows for transfer of maternal IgG to the fetus in the late stages of normal pregnancy, providing the major source of immunity for the neonate. It is also found in both human and rat intestinal lumens throughout both species' lifespan. Not intending to be bound by theory, the possibility of FcRN being the common denominator underlying our results must not be overlooked. Interestingly, the levels of human IgG seen in the fetal liver were essentially constant throughout all time points after injection.

The knowledge and insights provided by the above examples can serve as basis for more informed decisions when optimal dosing becomes a focal point in preclinical and, eventually, translational trials. The choice for IgG for this and the previous introductory experiment on TRAFIT was driven by the availability of variants of human IgG that bear no homology with rodents and that are detectable by readily available methods, as well as by its predominant role in fetal and neonatal immunity. Should other antibodies be amenable to TRAFIT, their pathway into the fetus, if at all present, may well be quite different, as each antibody class has specific receptor interactions, making their fetal transfer likely varied and unique. By capitalizing on the specific receptors and antibody types, TRAFIT could be used for targeted treatment of an array of medical and surgical diseases in the perinatal period. Regardless, the chronology of exogenous IgG kinetics after concentrated intra-amniotic injection hereby unveiled further suggests that TRAFIT may become a practicable strategy for the prenatal treatment of diseases such as select alloimmune disorders and infections.

### Example 5: Human Secretory IgA Kinetics in a Healthy Model of Transamniotic Fetal Immunotherapy (TRAFIT)

The transamniotic route has been recently shown experimentally to be a viable alternative for the administration of immunoglobulins to the fetus, though to date only with small molecules of the immunoglobulin-G class. We sought to determine whether secretory immunoglobulin-A (SIgA), a large conjugate of dimeric IgA not produced by the fetus responsible for mucosal immunity, is also amenable to transamniotic delivery, along with its routing kinetics, in a rodent model.

After IACUC approval, fetuses (n=94) from time-dated pregnant Sprague-Dawley dams (n=7) received intra-amniotic injections on gestational day 17 (E17, term=E21-22) of either saline (n=15) or a solution of 1 mg/mL of >95% homogeneous human SIgA (n=79) with a half-life of 5-6 days. Animals were euthanized at either E18 (n=13 fetuses), E19 (n=11), E20 (n=13), or E21 (n=42) for quantification of the IgA component by ELISA at gestational membranes, placenta, and select fetal anatomical sites. Saline controls were procured only at term E21 (n=13). Statistical analysis was by Mann-Whitney U-test with Bonferroni-adjusted significance.

Overall survival was 89.4% (84/94). None of the saline-injected animals had detectable human IgA, further confirming its lack of rodent homology. SIgA-injected fetuses showed human IgA in the stomach aspirate, intestinal wall, lungs, liver, and serum at all time points (figure). Overall, IgA levels were significantly higher in the gastric aspirate and in the intestine than in all other sites (p<0.001 for both), with intestinal levels remaining stable through E18-E21 (p=0.09-0.62 pairwise). Serum and placental levels were consistently low throughout, reaching near zero levels by E21.

The chronology of exogenous secretory-IgA kinetics after intra-amniotic injection was suggestive of fetal uptake by ingestion, leading to consistent levels in the gastrointestinal tract. Transamniotic fetal immunotherapy (TRAFIT) with secretory-IgA may become a novel strategy for enhancing early mucosal immunity and possibly contributing to the prevention of necrotizing enterocolitis.

The following methods were employed in Examples 1-3.

### Intra-Amniotic Injections

Nine pregnant Sprague Dawley dams (Charles River Laboratories, Inc., Wilmington, MA) were fed a normal diet ad libitum and housed individually under standard dark/light cycling conditions. On gestational day 18 (E18; term=E21-22) isoflurane (Patterson Veterinary, Greeley, CO), was used to induce general anesthesia via induction chamber inhalation at 4% and maintained at 2-2.5%, all in 100% oxygen. The average fetal rate weight was from about 4 to about 5 g. Using sterile technique, a midline laparotomy was made and the bicornuate uterus was eviscerated. Fetuses (n=109) received volume-matched (50 μL) intra-amniotic injections of either saline (n=29), or a suspension of IgG antibodies pooled from human serum (Sigma-Aldrich, St. Louis, MO), which was dissolved in saline at different concentrations, specifically: 5 mg/mL (n=28), 10 mg/mL (n=28), or 15 mg/mL (n=24). All fetuses from a given dam received identical injections. The human IgG was ≥95% homogeneous and previously tested by the manufacturer to lack homology with rodents. The saline group was added to further test such lack of homology. All injections were performed using a 33G non-coring needle on a 1004, syringe (both from Hamilton Company, Reno, NV) introduced into the amniotic cavity under direct visualization by the ventral aspect of the fetus, carefully avoiding it, the placenta, and the umbilical cord. Upon conclusion of the injections, the uterus was returned to the abdomen and the laparotomy was closed in 2 layers with 3-0 Vicryl (Ethicon, Somerville, NJ) and 5-0 Monocryl (Ethicon) simple running sutures. Animals were allowed to recover with Flagyl (Unichem Pharmaceuticals, Hasbrouck Heights, NJ) applied to the wound and analgesia by sustained release buprenorphine (Zoopharm, Windsor, CO).

### Specimen Procurement and ELISA

All dams were euthanized with chamber-inhaled carbon dioxide on E21.5. The midline incision was reopened allowing evisceration of the uterus. The myometrium was transected by each amniotic cavity and fetuses were removed with gestational membranes intact. Samples from peripheral blood, thymus, spleen, brain, and bone marrow were obtained. Maternal peripheral blood was also procured by cardiac puncture. The fetal thymus was obtained via fetal sternotomy, the spleen by laparotomy and brain by posterior craniotomy. Fetal blood was collected via subclavian cut down, allowed to clot, and then centrifuged at 2000 g for 10 min. followed by procurement of supernatant serum. The fetal bone marrow was procured from both femurs and tibias. Where the resulting specimen was too small, one of the humerus was also added. For all marrow procurements, the bones were transected both proximally and distally to expose the medullary cavity. They were then placed in 0.5 mL Eppendorf tubes previously punctured at their tip with an 18G needle, which were then placed inside a 1.5 mL Eppendorf tube; both tubes were centrifuged at 15RCF for 1 min. to isolate the bone marrow.

Once obtained, all specimens were washed with phosphate buffered saline and stored at −80° Celsius. At the time of analysis, samples from each site were standardized by weight and submitted to a human IgG Enzyme Linked Immunosorbent Assay (ELISA) (Abcam #195215, Cambridge, MA) in duplicate, as per manufacturer's recommendations. Concentrations were measured in ng/mL.

### Statistical Analysis

The duplicate concentrations of human IgG for each specimen were averaged to create a single data entry for each fetal tissue or serum sample. Medians and interquartile ranges were calculated for all specimen types for each of the three IgG concentrations and saline groups. Median regression was used to compare the groups and to evaluate for possible dose dependent relationships (Staffa S J and Zurakowski D. Calculation of Confidence Intervals for Differences in Medians Between Groups and Comparison of Methods. Anesth Analg 2020; 130 (2):542-546). Statistical significance was set at a two-tailed Bonferroni-adjusted p<0.008 to protect against Type I error from multiple comparisons between experimental groups (Staffa S J and Zurakowski D. Strategies in adjusting for multiple comparisons: A primer for pediatric surgeons. J Pediatr Surg 2020; 55 (9):1699-1705). Statistical power analysis indicated that the sample sizes for controls and experimental groups provided 80% power to detect 30% differences in human IgG median levels between groups to identify dose-response relationship for serum and different fetal tissues (nQuery Advisor version 7.0, Statistical Solutions, Cork, Ireland). Statistical analysis was performed using Stata software version 16.0 (StataCorp LLC, College Station, Texas).

The following methods were employed in Example 4.

### Intra-Amniotic Injections

Eight pregnant Sprague Dawley dams (Charles River Laboratories, Inc., Wilmington, MA) were fed a normal diet ad libitum and housed individually under standard dark/light cycling conditions. On gestational day 18 (E18; term=E21-22), isoflurane (Patterson Veterinary, Greeley, CO) was used to induce general anesthesia via induction chamber inhalation at 4% and maintained at 2-2.5%, all in 100% oxygen. Using sterile technique, a midline laparotomy was made and the bicornuate uterus was eviscerated. All fetuses (n=78) received volume-matched (50 μL) intra-amniotic injections of a 15 mg/ml suspension of lyophilized IgG antibodies pooled from human serum (Sigma-Aldrich, St. Louis, MO), which was dissolved in saline. The human IgG was ≥95% homogeneous and previously tested by the manufacturer to lack homology with rodents. Injections were performed using a 33G non-coring needle on a 100 μL syringe (both from Hamilton Company, Reno, NV) introduced into the amniotic cavity under direct visualization by the ventral aspect of the fetus, carefully avoiding the fetus, the placenta, and the umbilical cord. Upon conclusion of the injections, the uterus was returned to the abdomen and the laparotomy was closed in 2 layers with 3-0 Vicryl (Ethicon, Somerville, NJ) and 5-0 Monocryl (Ethicon) simple running sutures. Animals were allowed to recover from anesthesia. with Flagyl (Unichem Pharmaceuticals, Hasbrouck Heights, NJ) was applied to the wound and analgesia was maintained by sustained release buprenorphine (Zoopharm, Windsor, CO).

### Specimen Procurement and ELISA

Dams were euthanized at three different time-points after the injections—either E19, E20, or E21—via chamber-inhaled carbon dioxide. Two dams were randomly assigned to each time-point. The midline incision was reopened allowing evisceration of the uterus. The myometrium was carefully transected at each amniotic cavity allowing for each individual fetus and its associated gestational membranes to be dissected from the myometrium and kept intact, i.e., delivered “en caul”. Samples of amniotic fluid, amnion, chorion, placenta, as well as fetal serum, liver, and stomach aspirate were procured from each fetus. While still en caul and without disruption of the gestational membranes, the amniotic fluid was obtained by aspiration with a 31G needle. The gestational membranes were then ruptured, and both the chorion and amnion were dissected off of the chorionic plate at the insertion of the umbilical cord into the placenta. The chorion and amnion were then separated via blunt dissection and washed with phosphate buffered saline (PBS), along with the placenta. Fetal blood was obtained via subclavian cut down, allowed to clot, and then centrifuged at 2000 g for 10 minutes followed by procurement of supernatant serum. A fetal midline laparotomy was used to procure the fetal liver, which in the process also exposed the fetal stomach, which was left in situ. A 31G needle was then used to aspirate stomach contents under direct visualization.

Immediately after procurement, all specimens were stored in 1.5 mL tubes and flash frozen with dry ice before being stored at −80° C. At the time of analysis, samples from each site were standardized by weight and submitted to a human IgG Enzyme Linked Immunosorbent Assay (ELISA) with <3% cross reactivity with rodent IgG (Abcam #195215, Cambridge, MA) in duplicate, as per manufacturer's recommendations. Concentrations were measured in ng/mg.

### Statistical Analysis

The duplicate concentrations of human IgG for each specimen were averaged to create a single data entry for each fetal annex, tissue, or serum sample. Medians and interquartile ranges were calculated for all specimen types, for each of the three time-points (E19, E20 and E21). Two analyses were performed. The Mann-Whitney U test compared the median IgG detected in each sample across the three time-points. Median regression was performed to control for dam of origin for each fetal data point by nesting them based on maternal groups. A similar two-part analysis was applied to compare the amount of IgG between organ samples at each time-point. Statistical significance was set at a two-tailed Bonferroni-adjusted p<0.017 to protect against Type I error due to multiple comparisons between experimental groups. Statistical power analysis indicated that the sample sizes for controls and experimental groups provided 80% power to detect 30% differences in human IgG median levels between groups at different fetal tissues (nQuery Advisor version 7.0, Statistical Solutions, Cork, Ireland). Statistical analysis was performed using Stata software version 16.0 (StataCorp LLC, College Station, Texas).

### OTHER EMBODIMENTS

From the foregoing description, it will be apparent that variations and modifications may be made to the invention described herein to adapt it to various usages, antibody classes/types, and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for reducing a perinatal infection or risk thereof in a fetus or neonate, the method comprising administering to the amniotic fluid surrounding the fetus an antibody or antigen binding fragment thereof, wherein the fetus or neonate has or has a propensity to develop a perinatal infection, thereby reducing the infection or risk of infection.

2. The method of claim 1, wherein the infection is a viral infection or a microbial infection.

3. The method of claim 1, wherein the infection a cytomegalovirus (CMV), erythrovirus B19, Enteroviruses, hepatitis B virus, toxoplasmosis, rubella, herpes virus, varicella zoster virus (VZV), and/or human immunodeficiency virus (HIV) infection.

4. The method of claim 1, wherein the infection is a Bacteroides spp., Borrelia spp., Campylobacter spp., Chlamydia spp., Candida spp., Coccidioides spp., Enterococcus spp., Escherichia coli, Haemophilus spp., Listeria spp., Mycobacterium spp., Neisseria spp., Proteus spp., Salmonella spp., Staphylococcus spp., Streptococcus spp., Toxoplasma spp., Treponema spp., and/or Veillonella spp. infection.

5. The method of claim 1, wherein the administration is associated with a reduction in incidence of a surgical infection in the neonate.

6. The method of claim 5, wherein the surgical infection is necrotizing enterocolitis.

7. The method of claim 1, wherein the administration is associated with a reduction in incidence of neonatal infection and/or severity of infection.

8. A method for treating an alloimmune disorder in a developing fetus, the method comprising: administering to the amniotic fluid surrounding a developing fetus an antibody or antigen binding fragment thereof, thereby treating the alloimmune disorder.

9. The method of claim 8, wherein the alloimmune disorder is a hemolytic disorder.

10. The method of claim 1, wherein the antibody is administered by intra-amniotic injection.

11. The method of claim 1, wherein the antibody is derived from an IgA, IgD, IgE, IgG, IgM, IgY and/or IgW antibody.

12. The method of claim 1, wherein the antibody is a human antibody.

13. The method of claim 1, wherein the fetus is a mammalian fetus.

14. The method of claim 13, wherein the fetus is a human fetus.

15. The method of claim 1, wherein the antibody is administered prior to 5 weeks of pregnancy or after 10 weeks of pregnancy.

16. The method of claim 1, wherein from about 25 ng to about 500 mg of the antibody is administered

17. The method of claim 1, wherein the antibody comprises an Fc domain comprising one or more mutations at one or more positions corresponding to Thr 250, Met 252, Ser 254, Thr 256, Thr 307, Glu 380, Met 428, His 433, and/or Asn 434 of human IgG1.

18. The method of claim 1, wherein the administration is associated with the presence of and/or an increase in levels of the antibody in the serum of the fetus.

19. The method of claim 1, wherein the administration is associated with the presence of and/or an increase in levels of the antibody in a tissue of the fetus.

20. A kit suitable for use in carrying out the method of claim 1, wherein the kit comprises the antibody of claim 1.

21. The kit of claim 20, wherein the antibody is derived from human IgG, IgA, IgM, IgD, IgE, IgY, or Igga.
